(19) United States
(12) Patent Application Publication
(10) Pub. No.: US 2004/0147984 A1
(43) Pub. Date: Jul. 29, 2004

(54) Methods and apparatus for delivering low power optical treatments

(75) Inventors: Gregory B. Altshuler, Wilmington, MA (US); Joseph P. Caruso, Reading, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: PALOMAR MEDICAL TECHNOLOGIES, INC., Burlington, MA

(21) Appl. No.: 10/702,104

(22) Filed: Nov. 4, 2003

Related U.S. Application Data
(63) Continuation-in-part of application No. 09/996,662, filed on Nov. 29, 2001, now Pat. No. 6,648,904.

(51) Int. Cl. A61N 5/06

(52) U.S. Cl. 607/88; 607/89

(57) ABSTRACT

An apparatus is disclosed that uses at least one low power optical radiation source in a suitable head which can be held over a treatment area for a substantial period of time or can be moved over the treatment area a number of times during each treatment. The apparatus, a hand held light emitting applicator (LEA) or light emitting skin applicator (LESA), can be in the form of a brush or roller adapted to be moved over the patient's skin surface as radiation is applied to the skin. The skin-contacting surface of the LEA or LESA can have protuberances such as projections or bristles that can massage the skin and deliver radiation. In addition, an apparatus which delivers optical radiation to a treatment area is disclosed that contains a retrofit housing adapted to be joined to a skin-contacting device.

### PRIORITY

This application is a continuation-in-part of U.S. application Ser. No. 09/996,662 filed Nov. 29, 2001.

### BACKGROUND OF THE INVENTION

This invention relates to methods and apparatus for utilizing optical radiation to treat various dermatology, cosmetic, health, and immune conditions, and more particularly to such methods and apparatus operating at power and energy levels so low that they are safe enough and inexpensive enough to be performed in both medical and non-medical settings, including spas, salons and the home.

Optical radiation has been used for many years to treat a variety of dermatology and other medical conditions. Such treatments have generally involved utilizing a laser, flashlamp or other relatively high power optical radiation source to deliver energy to the patient's skin surface in excess of 100 watts/cm², and generally, to deliver energy substantially in excess of this value. The high-power optical radiation source(s) required for these treatments (a) are expensive and can also be bulky and expensive to mount; (b) generate significant heat which, if not dissipated, can damage the radiation source and cause other problems, thus requiring that bulky and expensive cooling techniques be employed, at least for the source; and (c) present safety hazards to both the patient and the operator, for example, to both a person's eyes and non-targeted areas of the patient's skin. As a result, expensive safety features must frequently be added to the apparatus, and generally such apparatus must be FDA approved and operated only by medical personnel. The high energy at the patient's skin surface also presents safety concerns and may limit the class of patients who can be treated; for example, it may often not be possible to treat very dark-skinned individuals. The high energy may further increase the cost of the treatment apparatus by requiring cooling of tissue above and/or otherwise abutting a treatment area to protect such non-target tissue.

The high cost of the apparatus heretofore used for performing optical dermatology procedures, generally in the tens of thousands of dollars, and the requirement that such procedures be performed by medical personnel, has meant that such treatments are typically infrequent and available to only a limited number of relatively affluent patients. However, the conditions for which such treatments can be useful are conditions experienced by most of the world's population. For example, such treatments include, but are not limited to, hair growth management, including limiting or eliminating hair growth in undesired areas and stimulating hair growth in desired areas, treatments for PFB, vascular lesions, skin rejuvenation, anti-aging including improving skin texture, pore size, elasticity, wrinkles and skin lifting, improved vascular and lymphatic systems, improved skin moistening, acne, removal of pigmented lesions, repigmentation, tattoo reduction/removal, psoriasis, reduction of body odor, reduction of oiliness, reduction of sweat, reduction/removal of scars, skin anti-aging, prophylactic and prevention of skin diseases, including skin cancer, improvement of subcutaneous regions, including fat reduction and cellulite reduction, pain relief biostimulation for muscles, joints, etc. and numerous other conditions (hereinafter sometimes collectively referred to as “patient conditions” or “conditions”). It would therefore be desirable if methods and apparatus could be provided, which would be inexpensive enough and low enough in both power and energy so that such treatments could be economically and safely performed by non-medical personnel, and even self-administered by the person being treated, permitting such treatments to be available to a greatly enlarged segment of the world's population.

### BRIEF SUMMARY OF THE INVENTION

The present invention provides methods and apparatus for utilizing optical radiation to treat various conditions at power and energy levels that are safe and inexpensive. An apparatus is disclosed that uses at least one low power optical radiation source in a suitable head which can be held over a treatment area for a substantial period of time or can be moved over the treatment area a number of times during each treatment. The apparatus, a hand held light emitting applicator (LEA) or light emitting skin applicator (LESA), can be in the form of a brush or roller adapted to be moved over the patient's skin surface as radiation is applied to the skin. The skin-contacting surface of the LEA or LESA can have protuberances such as projections or bristles that can massage the skin and deliver radiation. In addition, an apparatus which delivers optical radiation to a treatment area is disclosed that contains a retrofit housing adapted to be joined to a skin-contacting device.

In one embodiment, an apparatus for treatment of a patient condition is disclosed having an applicator with a skin-contacting surface comprising at least one protuberance, and at least one optical radiation source coupled to the applicator in a manner so as to, when activated, deliver optical radiation through the skin-contacting surface to a patient's skin in contact with the surface. The applicator can be in the form of a brush or roller adapted to be moved over the patient's skin surface as radiation is applied thereto. The applicator can be a hand-held unit. The skin-contacting surface can have at least one protuberance, such as projections and bristles, extending therefrom. The protuberance is adapted to apply a compressive force to the skin during use. The skin contacting end of each protuberance can have total internal reflection for the radiation when not in contact with the patient's skin, but passes radiation to the patient's skin when in contact therewith. The apparatus can also include a mechanism for applying a substance to the patient's skin as the skin is being irradiated.

In one embodiment, the at least one optical radiation source can be an array of optical radiation sources, each said source being mounted to deliver optical radiation through at least one corresponding protuberance. Each of the plurality of sources can be mounted to deliver radiation through a corresponding protuberance. At least one optical radiation source can be an array of semiconductor radiation-emitting elements. At least one optical radiation source can be operable at different wavelengths to effect a desired treatment protocol. At least one optical radiation source can be a continuous wave radiation source. The radiation sources can be retrofitted to the applicator, and can include a mechanism for attaching the sources to the applicator. Alternatively the at least one radiation source can be a part of the applicator.

The apparatus can further include a heat sink. In addition, the apparatus can include a handle, which is adapted to be held by the operator when the apparatus is in use, the heat sink sinking heat from at least one radiation source to the handle, heat from the handle being sinked to the operator's hand. In another embodiment the apparatus further includes a detector of contact between the applicator and the patient's skin, and controls operative in response to the detector for permitting radiation to be applied from the at least one source to the patient's skin.

In yet another embodiment, the skin-contacting surface is formed of a plate having good thermal conducting properties. The at least one optical radiation source can be mounted to the plate so that heat from the at least one source heats the plate. The heated plate is thereby adapted to heat a skin region during use. The apparatus can include a heat sink component in thermal contact with the at least one source, wherein the component is adapted to be cooled prior to use of the apparatus. The component can undergo a phase change when cooled, and returns to its initial phase when extracting heat from the at least one source.

In another aspect of the invention, a method for ameliorating a patient condition is disclosed in which a patient condition that is normally responsive to a known power density of phototherapeutic radiation is selected and a series of temporally spaced treatment sessions is delivered to a patient, where each session provides a power density of therapeutic radiation lower than typical power density needed to treat the patient condition in medical environments. The method can comprise the steps of selecting a patient condition normally responsive to a known power density of phototherapeutic radiation, and delivering a series of temporally spaced treatment sessions to a patient. Each session provides a power density of therapeutic radiation lower than the typical power density needed to treat the patient condition. The series of temporally spaced treatment sessions can be continued until the patient condition is ameliorated by a cumulative effect of the series of treatment sessions. The power density applied to the patient's skin surface is between approximately 1 mW/cm² and approximately 100 W/cm², and depends at least on the condition being treated and the wavelength of the radiation. Preferably, the energy at the patient's skin surface is between 10 mW/cm² and 10 W/cm². The radiation can be applied for a duration of one second to one hour. The method can use a power density for the series of treatment sessions delivered to the patient that is determined by the equation:

P(N)=P(1)/σ(N, ΔT, β), wherein

P(1) is the known power density for a single treatment, N is the number of treatments, ΔT is a temperature rise of tissue or cells undergoing treatment with P(1), β is a ratio of treatment time with P(N) to treatment time with P(1), and σ is as follows:

$$σ ( N , {τ}_{1} , {τ}_{N} , G ) := \frac{\frac{E}{R \cdot ln ( \frac{A \cdot {τ}_{1}}{G} )} - 310 \cdot K}{\frac{E}{R \cdot ln ( \frac{A \cdot {τ}_{N} \cdot N}{G} )} - 310 \cdot K} \cdot \frac{1 - exp ( \frac{- {τ}_{N}}{TRT} )}{1 - exp ( \frac{- {τ}_{1}}{TRT} )}$$

wherein A=3.1×10⁹⁸ s⁻¹, E is 150000 J/mol, and R is 1.986 J/mol·K.

In one embodiment, the method includes moving a head containing a source for the optical radiation over the patient's skin surface as the radiation is being applied thereto. The rate at which the head is moved over the skin surface and the number of times the head is passed over a given area of the patient's skin surface is such that the dwell time over each given area is within the duration. The optical radiation applied during the applying step can be continuous wave radiation.

In another embodiment, the method includes moving a head containing a source of the radiation over the patient's skin surface as the radiation is being applied thereto. The head can have a skin contacting surface which cleans and/or abrades the patient's skin surface as the head is moved thereover. The optical radiation applied during the applying step can be continuous wave radiation. The frequent intervals are approximately from several times per day to monthly treatments. Another feature of the present invention is that other treatments can be combined with the skin treatment, such as hygiene habits (i.e., showering, bathing, shaving, brushing one's teeth, etc.), mechanical and electrical massaging, stimulation, heat or cold therapy, topical drug or lotion therapy, and acupuncture therapy.

The condition being treated can be one of the conditions listed in Table 1, and the wavelength of the radiation can be within the corresponding range indicated in Table 1. The source of the radiation operates in a wavelength and/or a wavelength band suitable for treating dermatology, cosmetic or health conditions. The source can be an array of radiation sources, wherein the sources are operable at different wavelengths to effect a desired treatment protocol.

The method of the present invention can further include sinking heat from a source of the radiation. The source can be in an applicator having a handle held by an operator, wherein the sinking heat includes sinking heat from the source to the handle and wherein heat from the handle being sinked to the operator's hand. A source of the radiation can also be in an applicator having a skin-contacting surface. Pressure can be applied to the skin contacting surface to enhance the efficiency of energy delivery from the source. The pressure can cause projections from the skin contacting surface to compress the patient's skin.

In yet another embodiment, the method of the present invention can include utilizing a source of radiation that is in an applicator that has a skin-contacting surface. The skin contacting surface can have optical projections and/or bristles that extend from the surface. The optical projections/bristles can be used to concentrate optical radiation from the suitable radiation source.

The method of the invention can further include one of cooling and freezing an applicator containing the suitable radiation source prior to performing the applying step. The source of the radiation can be coupled to an applicator having a skin-contacting surface or points as in brush. The method can include detecting contact of one of the skin-contacting surface and projections/bristles extending from the surface with the patient's skin, and permitting delivery of optical radiation from the suitable radiation source to the patient's skin in response to the detection. Alternatively, a source of the radiation can be coupled to an applicator having a skin-contacting surface. The applicator can be adapted to apply a lotion to the patient's skin during at least a portion of the applying step. The source of the radiation can also be in an applicator having a skin-contacting surface, wherein the method is being applied for skin rejuvenation, and wherein during the applying step, the applicator abrades dead skin from the patient's skin surface while the applied optical radiation is facilitating collagen regrowth.

In another embodiment, the method of the present invention can further include radiation that is simultaneously delivered to a plurality of spaced small spots on the patient's skin to heat the spots. The method can further including applying a substance to the patient's skin and heating the spots to facilitate delivery of at least a portion of the substance to the patient's body through the heated spots. The delivery of the radiation can be combined with at least one of vibrating or otherwise stimulating the skin, magnetic field, electric field and acoustic field. It is also possible that retroreflecting light energy can exit the patient's skin back into the skin.

In one aspect of the invention, a method for ameliorating a patient condition is disclosed in which optical radiation is applied to penetrate into a target region of a patient's skin and the target region is agitated while applying the optical radiation, whereby the optical path of the radiation is varied during treatment to effect as larger volume within the target region.

A method is also provided for ameliorating a patient condition in which optical radiation is applied to penetrate into a target region of a patient's skin and the surface of the target region is abraded prior to, or during, application of the optical radiation, whereby surface obstructions to the radiation can be removed to effect as greater penetration within the target region.

In yet another aspect, the invention provides an apparatus for treatment of a patient condition comprising light emitting applicator (LEA) or light emitting skin applicator (LESA) having an output surface, which can either directly contact skin or can apply a substance directly to the skin, such as lotion, gel, layer or optically transparent material or spacing. At least one optical radiation source is coupled to the applicator in a manner so as to, when activated, deliver light through the skin contacting surface to the patient's skin in contact with the surface, the at least one radiation source being selected and the applicator being designed so as to deliver optical radiation having an energy at the patient's skin surface which is insufficient to have any appreciable therapeutic effect during a single treatment. The at least one radiation source can be selected and the applicator can be designed so as to deliver optical radiation in a series of temporally spaced treatment sessions to the patient, where each session provides a power density of a therapeutic radiation lower than a typical power density needed to treat the patient condition. The series of temporally spaced treatment sessions have a cumulative effect resulting in the amelioration of the patient condition. The energy at the patient's skin surface can be between approximately 1 mW/cm² and approximately 100 W/cm², the energy applied depending at least on the condition being treated and the wavelength of the radiation. The energy at the patient's skin surface is preferably between 10 mW/cm² and 10 W/cm².

The applicator can be in the form of a brush adapted to be moved over the patient's skin surface as radiation is applied thereto. The skin contacting surface can have projections and/or bristles extending therefrom. The at least one optical radiation source can be an array of optical radiation sources, each the source being mounted to deliver optical radiation through a corresponding one or more projections or bristles. The skin contacting end of each projection/bristle can have total internal reflection for the radiation when not in contact with the patient's skin, but passes radiation to the patient's skin when in contact therewith.

In another embodiment of the invention, the applicator can contact the treatment area, with high friction, through an optically transparent layer. The applicator can be pressed up against the skin such that it contacts the skin at or near a target area. The applicator can be mechanically agitated in order to treat the subsurface organs or other biological structures without moving the applicator from the contact area. For example, an applicator can be pressed up against a patient's cheek, such that the applicator contacts the patient's cheek at a contact area. The applicator can be massaged into the patient's cheek to treat the patient's teeth or underlying glands or organs while the physical contact point on the surface of the skin remains unchanged.

In yet another embodiment of the invention, a light emitting applicator can be attached or incorporated into an existing skin applicator, such as skin brushes, shower brushes, shave brushes, tooth brushes, razors, microabrasing applicators, massage devices, sponges, lotions, gels, soaps, topical drug distributors, and heat or cold applicators.

In one embodiment, the at least one optical radiation source is an array of optical radiation sources. The array of sources can be in a semiconductor wafer mounted on a heat sink. The wafer can be designed as a matrix or an array of light emitting diode or vertical surface emitting diode lasers. The sources can be operable at different wavelengths to effect a desired treatment protocol. The at least one optical radiation source can be a continuous wave radiation source or can be a pulsed radiation source with frequency high enough to cover the treatment area.

In another embodiment, the apparatus can include a heat sink, which is capable of removing heat from light sources, power supply and other heat dissipation components inside the apparatus. The apparatus of the present invention can further include a handle for the apparatus, which is adapted to be held by the operator when the apparatus is in use, the heat sink sinking heat from the at least one radiation source to the handle, heat from the handle being sinked to the operator's hand.

In yet another embodiment, the apparatus can further include a detector of contact between the applicator and the patient's skin, and controls operative in response to the detector for permitting radiation to be applied from the at least one source to the patient's skin. The apparatus can further include a mechanism for protecting the patient's eyes and/or a portion of the treatment area or an area outside of the treatment area, such that an area that requires less or no treatment can be protected from potential injury.

The apparatus may also include a mechanism for applying a substance to the patient's skin as the skin is being irradiated. This substance can provide benefits for the skin and other parts of the human body, such as hair and nails. This substance can be activated by the apparatus for better delivery into the skin, glands, hair, nails and/or for enhancing the treatment effect of radiation.

The applicator can be a bath brush, wherein water can be applied through the applicator both for bathing and to cool the source(s). The water is applied through openings in the surface to form water streams. Radiation from the at least one source is also applied through the openings and the streams act as wave guides for delivery of the radiation to the patient. The applicator can also be shaped to fit a portion of the patient's body to be treated.

The apparatus of the present invention can further include a mechanism for vibrating and/or otherwise stimulating the skin. The apparatus may also include a mechanism for applying at least one of magnetic field, electric field and acoustic field to the patient's skin. In another embodiment, the invention further includes a generator activated by movement of the applicator over the patient's skin to generate electrical energy for the radiation sources.

The skin contacting surface of the present invention can be created such that it retroreflects radiation reflected from the patient's skin back into the skin. The radiation sources can be retrofitted to the applicator, and can include a mechanism for attaching the sources to the applicator. Preferably, at least one radiation source is part of the applicator. In a preferred embodiment, the applicator is a hand-held unit.

The skin-contacting surface can be formed of a plate having good thermal conducting properties. The optical radiation source(s) can be mounted to the plate so that heat sinked from at least one source heats the plate and the heated plate can heat the patient's skin with which it is in contact. In one embodiment, the invention can include a heat sink component in thermal contact with a source. The component can be adapted to be at least cooled prior to or during use of the apparatus. The heat sink or an associated element can undergo a phase change when cooled, and returns to its initial phase when sinking heat from the at least one source (e.g., to extract hear by melting or evaporation).

In another aspect of the invention, a method is disclosed for treating a patient condition by applying optical radiation from a suitable source to the patient's skin. The radiation can have an energy at the patient's skin surface of between approximately 1 mW/cm² and approximately 100 W/cm², wherein the energy applied depends at least on the condition being treated and the wavelength of the radiation. The energy at the patient's skin surface is preferrably between 10 mW/cm² and 10 W/cm². The radiation can be applied for a duration of one second to one hour.

In yet another aspect, the present invention provides a method for treating a dermatology, cosmetic or health condition of a patient by applying low energy optical radiation from a suitable source to the patient's skin while simultaneously cleaning/abrading the patient's skin. Special lotions with chemical or abrasive properties can provide these benefits.

In other aspects, the present invention provides methods and apparatus to treat patients using the applicator of the present invention in combination with a lotion that contains a marker, such that the apparatus can work only if the marker is on the treatment area. The method for treating dermatology, cosmetic and health conditions of a patient is substantially as shown and described herein.

In another embodiment, an apparatus for treatment of a patient condition is disclosed having an applicator including at least one liquid delivery conduit for directing liquid onto a skin surface, and at least one optical radiation source coupled to the applicator in a manner so as to, when activated, deliver optical radiation together with the liquid to the skin surface. The applicator can be hand-held. The applicator can be a bath brush, wherein water can be applied through the applicator both for bathing or showering. Water can be applied to also cool at least one radiation source. Water can also be applied through openings in the surface to form water streams. Radiation from the at least one source can also be applied through the openings, so that the streams can act as wave guides for delivery of the radiation to the patient. The applicator can be shaped to fit a portion of the patient's body to be treated. The apparatus can include a mechanism for vibrating and/or otherwise stimulating the skin. The radiation sources can be retrofitted to the applicator, and can include a mechanism for attaching the sources to the applicator. The radiation source can also be a part of the applicator.

The skin-contacting surface can be formed of a plate having good thermal conducting properties. At least one optical radiation source can be mounted to the plate so that heat extracted from at least one source heats the plate. The heated plate thereby is adapted to heat a skin region during use. The apparatus can further include a heat sink component in thermal contact with at least one source, wherein the component is adapted to be cooled prior to use of the apparatus. The component can undergo a phase change when cooled, and can return to its initial phase when sinking heat from at least one source.

In another embodiment, an apparatus for treatment of a patient condition is disclosed having an applicator with a skin-contacting surface, and at least one optical radiation source coupled to the applicator in a manner so as to, when activated, deliver optical radiation through the skin-contacting surface to a patient's skin in contact with the surface. The apparatus further comprises a mechanism for applying at least one of a magnetic field, an electric field and an acoustic field to the patient's skin. The applicator can be a hand-held unit. The skin contacting surface can be created such that it retro-reflects radiation reflected from the patient's skin back into the skin. The apparatus can include a generator activated by movement of the applicator over the patient's skin to generate electrical energy for the radiation sources. The radiation sources can be retrofitted to the applicator, and can include a mechanism for attaching the sources to the applicator. At least one radiation source can be part of the applicator.

The skin-contacting surface of the applicator can be formed of a plate having good thermal conducting properties, wherein at least one optical radiation source is mounted to the plate so that heat extracted from the at least one source heats the plate. The applicator can further include a heat sink component in thermal contact with at least one source, wherein the component is adapted to be cooled prior to use of the apparatus. The component can undergo a phase change when cooled, and can return to its initial phase when sinking heat from said at least one source.

In yet another embodiment, an apparatus for treatment of a patient condition is disclosed having a retrofit housing adapted to be joined to a skin-contacting device, and at least one optical radiation source coupled to the retrofit housing in a manner so as to, when activated, deliver optical radiation to a skin surface concurrently with use of the skin-contacting device. The skin-contacting device can be in the form of a brush or roller adapted to be moved over the patient's skin surface as radiation is applied thereto. The skin-contacting surface can have at least one protuberance, such as projections and bristles extending therefrom, that are adapted to apply a compressive force to the skin during use. At least one optical radiation source can be an array of semiconductor radiation-emitting elements. At least one optical radiation source can be operable at different wavelengths to effect a desired treatment protocol.

### BRIEF DESCRIPTION OF THE DRAWINGS

FIG. 1 is a simplified schematic sectional view of an applicator head, according to the invention, having a flat skin-contacting surface;

FIG. 2 is a schematic sectional view of an alternative head in which bristles are used to deliver light from the radiation sources in wafer/package to the patient's skin;

FIG. 3 is a schematic sectional view of a head in which projections are used to deliver light from the radiation sources in wafer/package to the patient's skin;

FIG. 4 is a graph of the Arrhenius integral showing η as a function of the number of treatments;

FIG. 5A is a schematic illustration of the total internal reflection phenomenon in which narrow divergence is normally completely reflected from distal end of projections;

FIG. 5B is a schematic illustration of the total internal reflection phenomenon when the distal end of projections contacts the skin;

FIG. 5C is a schematic illustration of the total internal reflection phenomena in which narrow divergence is normally completely reflected from distal end of transparent bristle;

FIG. 5D is a schematic illustration of the total internal reflection phenomena when the distal end of transparent bristles contacts the skin;

FIG. 6 is a schematic of a shower-head LEA;

FIG. 7 is a schematic of one example of a light emitting shaving brush;

FIG. 8 is schematic of high efficiency applicator with both photo and thermal effect;

FIG. 9 is a graph of the population of bacteria versus time for periodic treatments comparing high intensity treatment, few treatment method (1) to the low intensity, multiple dose treatment method of the present invention (2);

FIG. 10 is a graph of the light dose per treatment versus the number of treatments;

FIG. 11A is a top perspective of a roller device with a light projection system;

FIG. 11B is a sectional front view of the roller in FIG. 11A; and

FIG. 12A is a cross-sectional illustration of a hand-held light emitting device according to this invention;

FIG. 12B is a bottom-up view of a hand-held light emitting device according to this invention.

FIG. 13 is an illustration of another embodiment of the invention in which a retrofit or “snap-on” accessory phototreatment apparatus is joined to a skin surface treatment device; and

FIG. 14 is an illustration of another retrofit apparatus for use in connection with a showerhead.

### DETAILED DESCRIPTION OF THE INVENTION

The invention generally involves the use of a low power optical radiation source, or preferably an array of low power optical radiation sources, in a suitable head which is either held over a treatment area for a substantial period of time, i.e. one second to one hour, or is moved over the treatment area a number of times during each treatment. Depending on the area of the patient's body and the condition being treated, the cumulative dwell time over an area during a treatment can be within the ranges indicated. The apparatus used will sometimes be referred to as a hand held light emitting applicator (LEA) or light emitting skin applicator (LESA). The treatments may be repeated at frequent intervals, i.e. daily, or even several times a day, weekly, monthly or at other appropriate intervals. The interval between treatments may be substantially fixed or may be on an “as required” basis. For example, the treatments may be on a substantially regular or fixed basis to initially treat a condition, and then be on as an “as required” basis for maintenance. Treatment can be continued for several weeks, months, years and/or can be incorporated into a patient's regular routine hygiene practices.

Thus, while light has been used in the past to treat various conditions, such treatment has typical involved one to ten treatments repeated at widely spaced intervals, for example, weekly, monthly or longer. By contrast, the number of treatments for this invention can be from ten to several thousand, with intervals between treatments from several hours to one week or more. It has been demonstrated by the inventors, through experiments in vascular and pigmented lesions treatment with light, that multiple treatments with low power could provide the same effect as one treatment with high power. The mechanism of treatment can include photochemical, photo-thermal, photoreceptor, photo control of cellular interaction or some combination of these effects. For multiple systematic treatments, a small dose can be effective to adjust cell, organ or body functions in the same way as systematically using medicine.

Theoretically for a thermal shock response-type mechanism, the power density for N treatments P_{N} can be low compared with the power density for a single treatment P₁ while achieving the same biological results. Using the Arrhenius integral, the following equation has been determined for

σ(N, τ₁, τ_{N}, G)=P₁/P_{N}:

$$σ ( N , {τ}_{1} , {τ}_{N} , G ) := \frac{\frac{E}{R \cdot ln ( \frac{A \cdot {τ}_{1}}{G} )} - 310 \cdot K}{\frac{E}{R \cdot ln ( \frac{A \cdot {τ}_{N} \cdot N}{G} )} - 310 \cdot K} \cdot \frac{1 - exp ( \frac{- {τ}_{N}}{TRT} )}{1 - exp ( \frac{- {τ}_{1}}{TRT} )}$$

where

$$\begin{matrix}A := 3.1 \cdot {10}^{98} \cdot {s}^{- 1} & E := 150000 \frac{J}{mol} & R := 1.986 \frac{J}{mol \cdot K} ,\end{matrix} \qquad ( 1 )$$

G is the value of the Arrhenius integral after treatment, which is a measure of thermally dysfunction biomolecules in treated organ. τ₁ and τ_{N} are the treatment times of P₁ and P_{N}, respectively. TRT is thermal relaxation time of the treated organ.

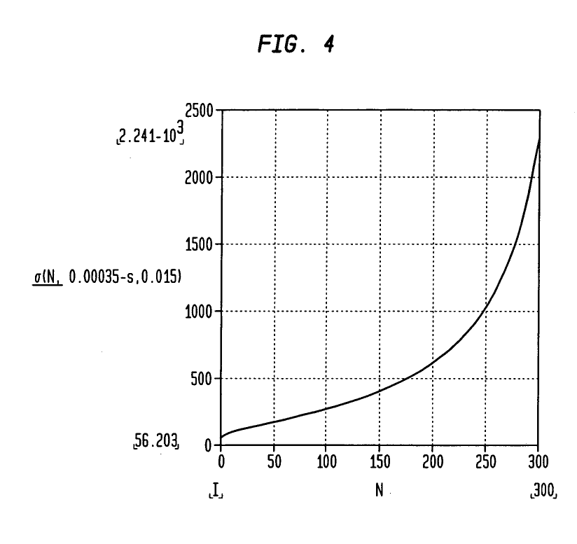

FIG. 4 shows σ(N, τ₁, τ_{N}, G) as function of the number of treatments for a target with TRT of 5 ms, which is typical for a small 90 microns blood vessel, τ₁ is 0.5 ms, which is typical treatment mode for selective thermolysis, when τ<<TRT, τ_{N} is 900 s (15 minute procedures), and G is 0.015. The graph shown in FIG. 4 suggests that power density for 140 treatments (one month of daily treatments) can be dropped by 70 times from that required for one treatment and can be dropped for 300 treatments (one year of daily treatments) by 2250 times. The relation between the number, frequency and length of treatments can be different for each condition, with the same tendency of requiring a lower power density when multiple, relatively closely spaced treatments are provided._For a given condition, the required power density or energy can also vary as a function of the wavelength or wavelength band used for the treatment.

Equation (1) and FIG. 4 can be used for estimation of treatment parameters for skin rejuvenation and wrinkle reduction by multiple treatments. A cosmetic improvement has been observed with an average value of 1.88 reduction in wrinkle appearance as measured on the Fitzpatrick Wrinkle Severity scale (Bjerring P., Clement M., Heickendorff L., Egevist H., Kiernan M.: Selective non—ablative wrinkle reduction by laser, J. Cutaneous Laser Therapy, 2000; 2: 9-15). This improvement was achieved with one treatment using dye lasers at 585 nm wavelength, 0.00035 s pulsewidth and 2.4 J/cm² fluence and 6900 W/cm² power density. As illustrated by equation (1) and FIG. 4, the same results can be achieved with daily 15 min treatments with 585 nm light sources with power density 50 W/cm² after one month and with power density 3 W/cm² after one year. Such parameters can be implemented into the light emitting applicator (LEA) proposed in the present invention with LEDs, diode lasers or lower power lamps as light sources. In addition, the number of treatments can be further reduced by simultaneously heating the skin to 38-42° C. This can be achieved using the same applicator or an external heating source.

Similarly, the fluence or power can be decreased using multiple treatments to achieve other photochemical effects on biological tissues. In one embodiment, the photochemical process treated with reduced fluence or power and multiple treatments is acne treatment with blue light (A. R. Shalita, Y. Harth, and M. Elman. “Acne PhotoClearing (APC™) Using a Novel, High-Intensity, Enhanced, Narrow-Band, Blue Light Source” Clinical Application Notes, V.9, N1]. Acne is a disease of the sebaceous gland in which the gland becomes plugged with sebum and keratinous debris as acne bacteria (i.e., Propionibacterium acnes or P. acnes) undergo abnormal proliferation. The destruction of P. acnes is the indispensable part of any effective acne therapy.

Being an effective method of acne treatment, APC is based on the fact that the acne bacteria produce porphyrins as a part of their normal metabolism process. Irradiation of porphyrins by the light causes a photosensitization effect that is used, for example, in the photodynamic therapy of cancer. The strongest absorption band of porphyrins is called the Soret band, which lies in the violet-blue range of the visible spectrum (405-425 nm). While absorbing photons, the porphyrin molecules undergo singlet-triplet transformations and generate the singlet atomic oxygen that oxidize the bacteria that injures tissues. The same photochemical process is initiated when irradiating the acne bacteria. The process includes the absorption of light within endogenous porphyrins produced by the bacteria. As a result, the porphyrins degrade liberating the singlet oxygen that oxidize the bacteria and eradicate the P. acnes to significantly decrease the inflammatory lesion count. The particular clinical results of this treatment are reported (A. R. Shalita, Y. Harth, and M. Elman. “Acne PhotoClearing (APC™) Using a Novel, High-Intensity, Enhanced, Narrow-Band, Blue Light Source” Clinical Application Notes, V.9, N1). In clinical studies, the 60% decrease of the average lesion count was encountered when treating 35 patients twice a week for 10 minutes with 90 mW/cm² and dose 54 J/cm² of light from the metal halide lamp. The total course of treatment lasted 4 weeks during which each patient underwent eight treatments.

Instead of using single or few treatments of intense light, which must be performed in a supervised condition such as a medical office, the same reduction of the bacteria population level can be reached using a greater number of treatments of significantly lower power and dose using the light emitting applicator (LEA) proposed in this invention. Such lower power treatment with LEA can be performed in the home environment. It should be noted that the relation between the number of treatments per a predefined period of time and the total change of the bacteria population level is not straightforward due to the complex population dynamics of the bacteria during the course of treatment. Thus, the user will normally not get successful results by shortening the inter-treatment time using this small dose per treatment method. This is explained below using the classical Verhulst model.

The Verhulst model suggests that the population growth rate is limited by the competition between individuals. Applying this model to the bacteria yields the following differential equation:

$$\frac{ⅆ}{ⅆ t} B = a B ( 1 - \frac{B}{{B}_{st}} ) , \qquad ( 2 )$$

where B is the bacteria population level at time t, Bₛₜ is the stationary population level, and α is the population growth rate in the absence of competition, i.e., for B<<Bₛₜ. Equation (2) is valid in between the light treatments. The solution for equation (2) reads:

$$B ( t ) = B ( 0 ) \cdot \frac{B ( 0 ) \cdot exp ( a t )}{1 + ( exp ( a t ) - 1 ) \cdot \frac{B ( 0 )}{{B}_{st}}} , \qquad ( 3 )$$

where B(0) is the initial population level.

The effect of the treatment must be accounted for by adding a new parameter, χ, into the right-hand side of equation 2, which describes the light-induced decrease of the population level. Intensity of light at the treatment site is W(t), where arbitrary time dependence is assumed. The light effect on a bacterium is described by the parameter, χ, that is, the eradication rate per unit light intensity and unit population level. Assuming the linear dependence of the eradication rate on the intensity and the population level, the governing differential equation assumes the form:

$$\frac{ⅆ}{ⅆ t} B = a B ( 1 - \frac{B}{{B}_{st}} ) - χ W ( t ) B . \qquad ( 4 )$$

Equation (4) presents some modification of the original Verhulst model. Like the original model, the above equations may be solved analytically.

Periodic treatments can also be modeled. Function W(t) is the periodic sequence of rectangular pulses. The time interval between pulses and the time delay before the first pulse is τ₁ and the pulse duration is τ₂. The period is given by τ=τ₁+τ₂. In the present case we are interested in the population level at the end of each pulse, i.e., at the time instant tₙ=n·τ, where n is the arbitrary pulse number ranging from 1. For α·τ₂<<1 the corresponding expression for bacteria population after η treatments Bₙ reads:

$${B}_{n} = \frac{B ( 0 ) \cdot exp [ n \cdot ( a τ - χ F ) ]}{1 + \frac{exp [ n \cdot ( a τ - χ F ) ] - 1}{exp ( a τ - χ F ) - 1} \cdot [ exp ( a τ ) - 1 ]} \qquad ( 5 )$$

with F=W·τ₂ is the light dose per treatment.

Through a comparison of the experimental data reported by Shalita, et al. (Clinical Application Notes, V.9, N1]. and model (5), we obtain the following values of the model parameters: a=0.3 weeks⁻¹, χ=0.013 cm²/J, and Bₛₜ=10⁵ colonies/cm². These parameters were applied to equation (5) to evaluate the population level against time. The results of this comparison are presented in FIG. 9 demonstrating that the experimental model of the present invention closely mimic that of the clinical data of Shalita et al. Curve 1 is the clinical data of Shalita et al. in which 10 minutes with 90 mW/cm² and dose 54 J/cm² of light from the metal halide lamp was used. Curve 2 demonstrates daily treatments according to the present invention light emitting applicator (LEA) using 10 minutes with 13 mW/cm² and dose 7.8 J/cm² of light LED with wavelength 410-420 nm. The population level abruptly falls during treatments and grows slowly between the treatments. FIG. 9 demonstrates that with low power (13 mW/cm²) daily treatment with handheld light emitted applicator (LEA) proposed in present invention the same effect on bacteria can be achieved as with ClearLight™ high power (90 mW/cm²) stationary 192 lb. device (commercially available from Lumenis Inc. Santa Clara, Calif.).

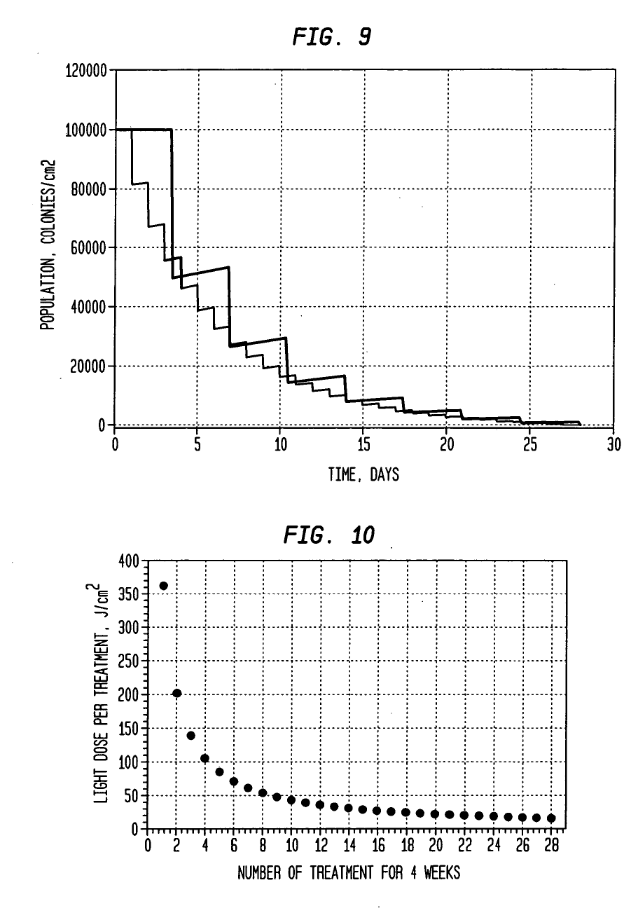

FIG. 10 is a graph demonstrating the amount of treatments needed with various light doses over a 4 week span in order to achieve identical bacteria reduction. For example, the dose for approximately 28 treatments is 24 times lower than for one treatment. The effects of acne treatment using the method of the present invention, can be enhancing using the following techniques. Compression of the skin can lead to better penetration of light to the sebaceous follicle including the gland. Optimal combination of different wavelengths from 400-700 nm range can be used. Longer wavelength can be more effective on sebaceous glands and can be used to regulate sebum production. The infundibulum and/or sebaceous gland can be heated. The optical treatment can be combined with cleaning of comedo and sebaceous follicle opening. The optical treatment can be used in combination with anti-bacterial and or anti-inflammatory lotions, which can be applied before and/or after optical treatment. The optical treatment can be used in combination with a lotion application containing a photo sensitizer. The optical treatment can be combined with a lotion application containing molecules that initiate photo sensitizer production as 5-aminolevulinic acid (ALA). Additionally, a lotion can be applied that contains absorption compounds, such as carbon, melanin, or a dye that increases light absorption resulting in better heating effects.

The specific light parameters and formulas of assisted compounds suggested in the present invention provide this treatment strategy. These treatments may preferably be done at home because of the high number of treatments and the frequent basis on which they must be administered, for example daily to weekly. As will be discussed later, various light based devices can be used to deliver the required light doses to a body. The optical radiation source(s) utilized may provide a power density at the patient's skin surface of from approximately 1 mwatt/cm² to approximately 100 watts/cm², with a range of 10 mwatts/cm² to 10 watts/cm² being preferred. The power density employed will be such that a single treatment will result in no appreciable therapeutic effect. Therapeutic effect can be achieved, as indicated above, by relatively frequent treatments over an extended time period. The power density will also vary as a function of a number of factors including, but not limited to, the condition being treated, the wavelength or wavelengths employed and the body location where treatment is desired, i.e., the depth of treatment, the patient's skin type, etc. A suitable source may, for example, provide a power of approximately 5-10 watts.

Suitable sources include semiconductor light emitters such as:

Light Emitting Diodes (LEDs) including edge emitting LED (EELED), surface emitting LED (SELED) or high brightness LED (HBLED). The LED can be based on different materials such as AlInGaN/AlN (emitting from 285 nm), SiC, AlInGaN, GaAs, AlGaAs, GaN, InGaN, AlGaN, AlInGaN, BaN, InBaN, AlGaInP (emitting in NIR and IR), etc. with lattice structure and others. Another suitable type of LED is an organic LED using polymer as the active material and having a broad spectrum of emission with very low cost.

Superluminescent diodes (SLDs). An SLD can be used as a broad emission spectrum source.

Laser diode (LD). A laser diode is the most effective light source (LS). A wave-guide laser diode (WGLD) is very effective but is not optimum due to coupling light into a fiber. Vertical cavity surface emitting laser (VCSEL) is most effective for fiber coupling for a large area matrix of emitters built based on a piece of wafer. This can be both energy and cost effective. The same materials used for LED's can be used for diode lasers.

Fiber laser (FL) with laser diode pumping.

Fluorescence solid-state light source with electric pumping or light pumping from LD, LED or current/voltage sources. The FLS can be an organic fiber with electrical pumping.

Other suitable low power lasers, mini-lamps or other low power lamps or the like may also be used as the source(s). LED's are the currently preferred radiation source because of their low cost, the fact that they are easily packaged, and their availability at a wide range of wavelengths suitable for treating the Conditions. In particular, MCVD technology may be used to grow a wafer containing a desired array, preferably a two-dimensional array, of LED's and/or VCSEL at relatively low cost. Solid-state light sources are preferable for monochromatic applications. However, a lamp, for example an incandescent lamp, fluorescent lamp, micro halide lamp or other suitable lamp is the preferable LS for white, red, NIR and IR irradiation.

Since the efficiency of solid-state sources is 1-50%, and the sources are mounted in very high-density packaging, heat removal from the emitting area is generally the main limitation on source power. For better cooling, a matrix of LS's can be mounted on a diamond, sapphire, BeO, Cu, Ag, Al, heat pipe, or other suitable heat spreader. The LS used for a particular apparatus can be built or formed as a package containing a number of elementary LS components. For improved delivery of light to skin from a semiconductor emitting structure, the space between the structure and the skin can be filled by a transparent material with a refractive index of about 1.3 or higher, without air gaps.

Light sources with mechanisms for coupling light into the skin can be mounted in or to any hand piece that can be applied to the skin, for example any type of brush, including a shower brush or a facial cleansing brush, massager, or roller (See, for example, U.S. application Ser. No. 09/996,662 filed Nov. 29, 2001, which is herein incorporated by reference in its entirety, for a device for controlling the temperature of the skin). In addition, the light sources can be coupled into a shower-head, a massager, a skin cleaning device, etc. The light sources can be mounted in an attachment that may be clipped, velcroed or otherwise affixed/retrofitted to an existing product or the light sources can be integrated into a new product.

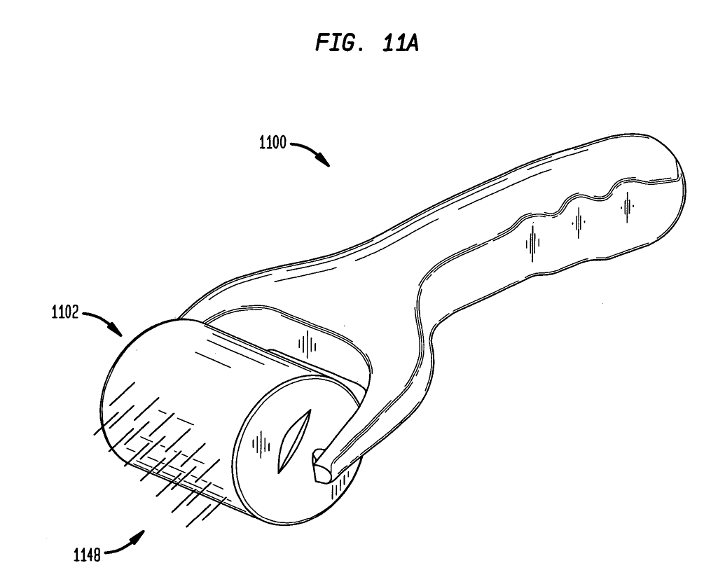

As shown in FIG. 11A, light sources 1102 can be attached to the outer surface of a roller assembly 1148 that can be used to control the temperature of the user as disclosed in U.S. application Ser. No. 09/996,662 filed Nov. 29, 2001, which is herein incorporated by reference. Alternatively, light sources 1102′ can project through the transparent outer surface of the roller assembly, which can be comprised of a transparent material with good heat transfer properties, such as sapphire or quartz or plastic. This can be achieved, for example, by replacing some of the channels 1118 with light sources as shown in FIG. 11B. Alternatively, light sources can be positioned on the interior of the roller 1112.

The sources utilized may generate outputs at a single wavelength or may generate outputs over a selected range of wavelengths or one or more bands of wavelengths. For a broadband source, filtering may be required to limit the output to desired wavelength bands. Where a radiation source array is employed, each or several sources may operate a selected different wavelengths or wavelength bands (or may be filtered to provide different bands), where the wavelength(s) and/or wavelength band(s) provided depend on the condition being treated and the treatment protocol being employed. Employing sources at different wavelengths may permit concurrent treatment for a condition at different depths in the skin, or may even permit two or more conditions to be treated during a single treatment. Wavelengths employed may be in the range from 290 nm to 20000 nm. Examples of wavelength ranges for various treatments will be provided later. The sources employed may also be continuous wave (CW), this term also including sources which are pulsed at a rate equal to or higher than 0.5 Hz, or can be a pulsed source operating at a suitable rate, for example 10 pulses per second to 10000 Hz. This rate can be synchronized with a biological repetition rate of the treated individual, for example with heart rate or breathing cycle, or may be synchronized with the rate of vibration of an acoustic wave being delivering to the body simultaneously with the light.

The head used for the treatment is preferably a brush-like apparatus with bristles extending from the head, which bristles are preferably optical fibers of organic or non-organic material through which the optical radiation is applied to the patient's skin, or the head may be a massage-like apparatus having pointed or rounded projections for contacting the skin and through which the optical radiation is applied to the patient's skin. In the case of a shower-head or other device for projecting water, the water can act as a wave guide for delivering the light to the patient's skin and no other type of coupler may be required. If a radiation source array is employed, it may be designed such that there is a radiation source over each projection, each bristle or each group of bristles. Where the contact portions of the bristles or projections do not transmit the light, the light is applied to the skin between and/or around the bristles/projections. The projections or bristles may clean the patient's skin to remove dead skin, dirt, bacteria and various treatment residue, and the projections or bristles may also stimulate and massage the skin, a process which facilitates various of the treatments. Projections and bristles can also concentrate the radiation to small spots on the skin surface, thereby substantially increasing the energy delivered to treatment spots for a given radiation source power and, particularly if pressure is applied to the head during treatment, can indent the patient's skin, bringing the applied radiation closer to the desired treatment or target area. The bristles or projections thus may significantly enhance the efficiency of energy delivery to a target area, permitting more effective treatment for a given source power. The source power, the spacing of the sources, the head design (i.e. the projections or bristles employed) and other apparatus parameters are selected so as to generate the energy or power density at the patient's skin surface previously discussed. The bristles employed may be harder or softer, or the shape of the projections may be adjusted, depending on the degree of abrasion desired for a particular treatment, the sensitivity of the patient's skin and other factors. A head having a uniform skin contacting surface which may be flat or curved, and may be smooth or abrasive, is also within the contemplation of the invention, although such head is not currently preferred at least because it does not concentrate the radiation to increase energy efficiency as does the projections/bristles.

The size of the head or brush employed can vary depending on the part of the body which the head is designed to treat, being, for example, larger to treat the body and smaller to treat the face. A larger body brush may for example be used as a bath brush, delivering both optical radiation and water to both clean the body as would a shower brush, while at the same time performing a light radiation treatment, for example, biostimulation. The water can also be used to cool the radiation sources. If the brush bristles are not optical fibers, the water can also act as a waveguide for the light being delivered to the patient's skin. The front part of the LEA that contacts the skin can be made from a soft material to prevent mechanical alteration. For example, it can be a brush with very small diameter flexible fibers or optical resin pad or elastic pad with optical channels.

While the low power radiation sources employed for this invention generate far less heat than the higher power sources previously employed, they do generate some heat, which, particularly for longer treatments, it is desirable to dissipate from the sources. A heat sink of a thermally conductive material, for example aluminum or some other metal or a thermally conductive ceramic, in contact with the sources can dissipate heat from the head, and heat can be removed from the heat sink into ambient air. Where the head has projections in contact with the patient's skin, these projections may be of a heat conducting material, permitting heat to be removed through the patient's body. This heat will not be high enough to cause pain or discomfort to the patient, and my cause mild hyperthermia of the patient's skin which may facilitate some treatments. Similarly, the heat sink may be extended to the apparatus handle, permitting heat through the heat pipe to be dissipated through the hand of the operator. Again, the heat will not be sufficient to cause any danger or discomfort. The applicator may also be placed in a refrigerator or freezer before treatment to provide mild hypothermia to the patient's skin during initial treatment and to facilitate heat removal from the radiation sources. For example, the heat sink may be a pack in contact with the sources which contains a freezable liquid, for example water, wax or other materials that have a melting temperature or evaporation temperature in the range suitable for cooling light sources and/or skin which undergoes a phase change as it is heated by the sources, the phase change resulting in significant heat removal. After treatment this material can be recycled back to the initial phase through the use of a special cooler or through cooling from ambient temperature. For example, this material can be wax or paraffin which has a melting temperature in the range between room temperature (20-30° C.) and tolerable skin temperature (38-42° C.).

The energy outputs from the apparatus indicated above are so low that, even if optical radiation from the apparatus was inadvertently shined on a person's eyes, it should cause no immediate injury to the person's eyes, and the person would experience discomfort causing them to look away or move the radiation away from their eyes before any injury could occur. The effect would be similar to looking directly at a light bulb. Similarly, injury to a patient's skin should not occur at the energy levels of this invention even if the recommended exposure intervals are exceeded. Again, to the extent a combination of parameters might result in some injury under some circumstance, patient discomfort would occur well before any such injury, resulting in termination of the procedure.

Energy efficiency may be enhanced and safety improved, although as indicated earlier, safety is not an issue for the apparatus of this invention, by having the radiation sources activated only when the projections, bristles or other skin-contacting surface are in contact with the patient's skin or permitting an output therefrom only when there is such contact. This may provide an output only for projections/bristles in contact, so that, for example, some sources, associated with bristles/projections that are in contact, are on while other sources, associated with bristles/projections that are not in contact, are off, or any contact may result in all projections/bristles providing an output. A suitable pressure sensor may, for example, be provided at the proximal end of each bristle or bristle group, the corresponding radiation source being activated in response to the sensor output; one or more sensors may be provided which detect contact and activate all radiation sources in response thereto; or a bristle or other output window may have total internal reflection until the distal end thereof is in contact with the patient's skin, with light being output from the bristle/window only when there is such contact. The contact sensor can be mechanical, electrical, magnetic or optical. The device can be equipped with a sensor, which can provide information about treatment results: For example, a fluorescent sensor can be used to detect the fluorescence of protoporphrine in acne. As treatment progresses, the fluorescent signal would decrease. This, this method can be used to indicate when treatment should be complete.

While it is possible that the energy requirements for apparatus of this invention could be small enough that they could be operated for a reasonable number of treatments with a non-rechargeable battery, it is currently contemplated that a rechargeable battery or electromechanical generator activated by movement of the applicator, such as is currently used, for example, with an electric toothbrush, would be utilized. A suitable power supply connected to an AC line could also be used.

While a single brush-like applicator is used for preferred embodiments, this is not a limitation on the invention. For example, the applicator may be in the form of a face-mask or in a shape to conform to other portions of a patient's body to be treated, the skin-facing side of such applicator having projections, water jets or bristles to deliver the radiation as for the preferred embodiments. While such apparatus could be moved over the patient's skin, to the extent it is stationary, it would not provide the abrading or cleaning action of the preferred embodiments.

The head could also have openings through which a substance such as a lotion, drug or topical substance is dispensed to the skin before, during or after treatment. Such lotion, drug, topical substance or the like could, for example, contain light activated PDT molecules to facilitate certain treatments. The PDT or ALA like lotion could also be applied prior to the treatment, either in addition to, or instead of, applying during treatment. LEA can be used in conjunction with an anti-perspirant or deodorant lotion to enhance the interaction between the lotion and the sweat glands via photothermal or photochemical mechanisms. The lotion, drug or topical substance can contain molecules with different benefits for the skin and human health, such as skin cleaning, collagen production, etc.

Conditions treatable utilizing the teachings of this invention include at least most of the Conditions previously mentioned and the list of applications for these teachings will surely expand as experience with the teachings increases. Table 1 lists some of the applications for these teachings, along with suitable parameters for utilizing the teachings for each of these applications.

Considering some possible applications, for skin rejuvenation, the optical radiation can stimulate collagen growth. Projections with optimized microsurface profile or bristles moving over the skin can provide microabrasion by peeling or otherwise removing dead skin and causing micro-trauma to the skin which the light helps repair by collagen growth. Since the target area for this treatment is the papillary dermis at a depth of approximately 0.1 mm to 0.5 mm into the skin, and since water in tissue is the primary chromophore for this treatment, the wavelength from the radiation source should be in a range highly absorbed by water or lipids or proteins so that few photons pass beyond the papillary dermis. A wavelength band from 900 nm to 20000 nm meets these criteria. For sebaceous gland treatment, the wavelength can be in the range 900-1850 nm, preferable around peaks of lipid absorption as 915 nm, 1208 nm, 1715 nm. For treatment of acne, the light can, among other things, kill acne-causing bacteria, a wavelength band from 290 nm to 700 nm accomplishing this objective. Hair growth management can be achieved by acting on the hair follicle matrix to accelerate transitions or otherwise control the growth state of the hair, thereby accelerating or retarding hair growth, depending on the applied energy and other factors.

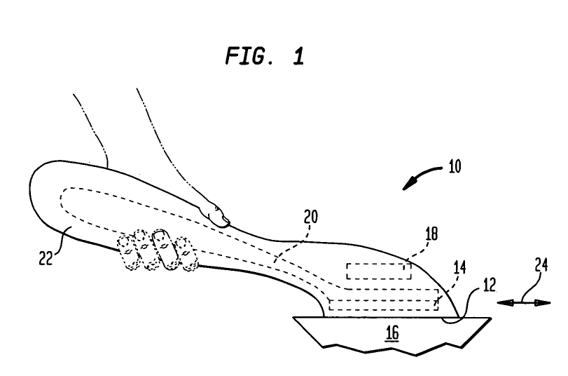

FIG. 1 is a semi-schematic sectional view of a simplified head 10 suitable for practicing the invention, this head having a flat skin-contacting surface, which may be smooth or abrasive. The skin-contacting surface 12 is preferably a layer, generally a thin layer, of a material which has a good optical match with skin, is optically transparent and preferably has good heat transfer properties, for example organic or mineral glass, dielectric crystal or sapphire. For better contact with skin, it can be flexible transparent plastic. A wafer or other suitable package 14 containing an array, for example a matrix array, of LED's or other suitable radiation sources is mounted in contact with layer 12 and directs radiation through this layer to the patient's skin 16. The radiation source array is driven from a suitable power source 18, which may, for example, include a rechargeable or disposable battery or a connection to a standard wall power plug, and also contains suitable controls, which may include manually operated controls, for turning the radiation sources on and off and for otherwise controlling operation thereof. While heat from the radiation sources may be sinked to the patient's skin 16 through layer 12, to the extent additional heat sinking is required, a heat sink or heat pipe 20 of suitable material having good heat transfer properties may be provided in thermal contact with wafer/package 14. Heat sink or heat pipe 20 is shown as extending into handle 22 so that heat may also be sinked into the hand of the operator. Alternatively, the heat sink/heat pipe 20 may be in contact with a container with a phase change transfer material such as ice or wax. Arrows 24 indicate two of the directions in which head 10 may be moved across the patient's skin 16. The head may also be moved in the directions in and out of the figure and in all other directions adjacent or parallel to the skin surface. If the spacing between the radiation sources and the patient's skin surface can be kept small enough, the light reaching the skin surface from each source can be fairly concentrated. Suitable optics in wafer/package 14, layer 12 or there-between can also be provided to concentrate the light from each source at the skin surface to enhance energy efficiency. A fly's-eye lens array may, for example, be employed for this function.

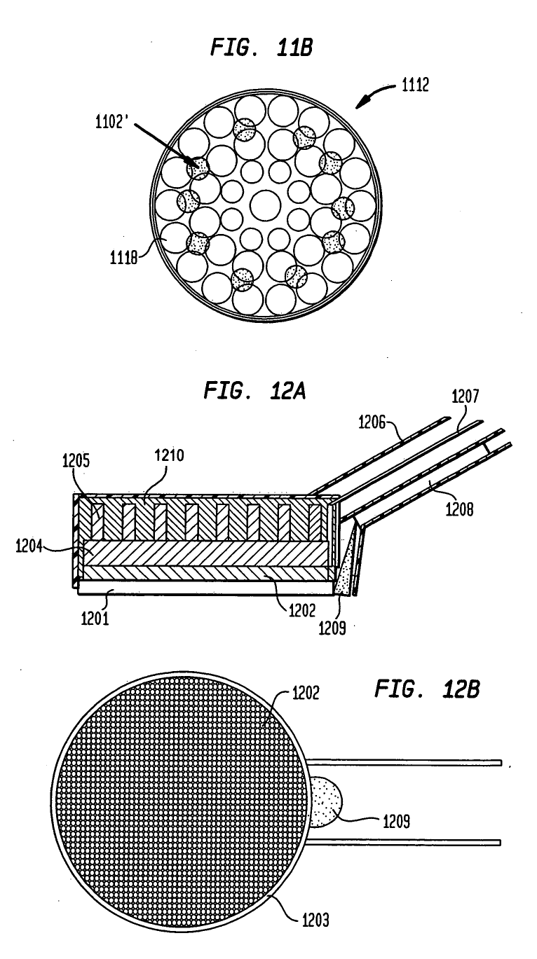

In another embodiment of the invention, the applicator can contact the treatment area, with high friction, through an optically transparent layer. The applicator can be pressed up against the skin such that it contacts the skin at or near a target area. The applicator can be mechanically agitated in order to treat the subsurface organs without moving the applicator from the contact area. For example, an applicator can be pressed up against a patient's cheek, such that the applicator contacts the patient's cheek at a contact area. The applicator can be massaged into the patient's cheek to treat the patient's teeth or underlying glands or organs while the physical contact point remains unchanged. As shown in FIGS. 12A and 12B, the headpiece 1203 of the applicator can contain a contact window 1201 composed of a transparent, heat transmitting material. The contact window 1201 can be adapted to be removable so that it can be replaced by the user. An array 1202 of LEDs or VCSELs or other light sources can be positioned such that the light from the array of light sources 1202 projects through the contact window 1201. A heatsink 1204 can be thermally coupled to the array of light sources 1202 and be held in place with heatsink pins 1205. The heatsink 1204 and heatsink pins 1205 can be in thermal contact with a material 1210 of high heat capacity or a phase change material, such as ice, water, wax or paraffin. The applicator can have a handle 1206 through which the power supply wire 1207 can be attached. Alternatively, the handle 1206 can have an internal power supply, such as a battery. A lotion cartridge 1208 can be located within the handle 1206 such that lotion can be stored and can flow to the skin through the lotion outlet 1209.

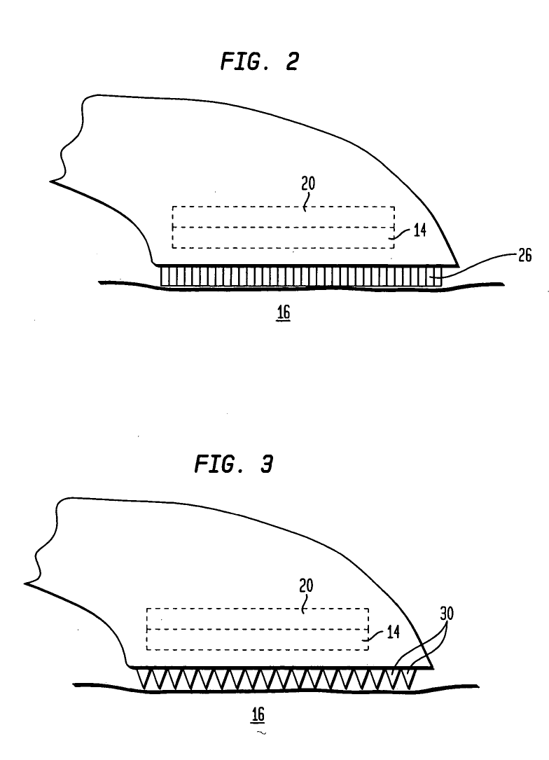

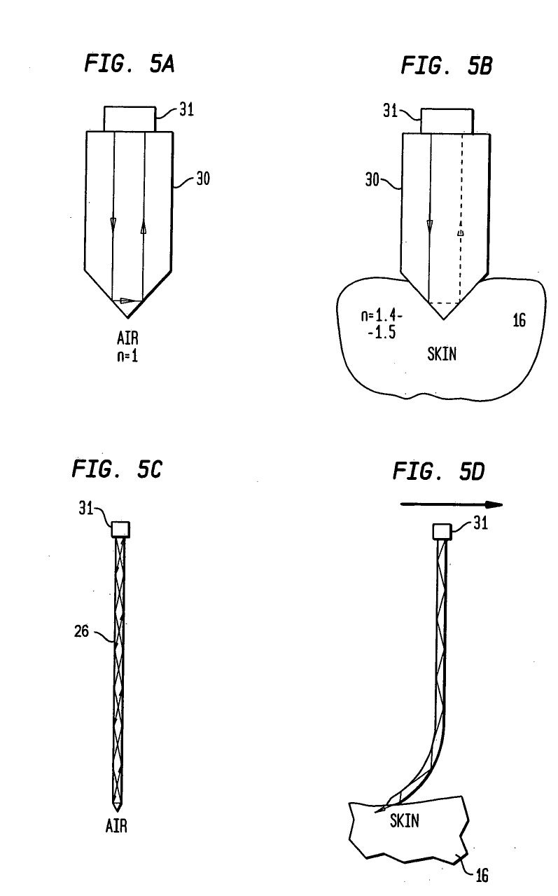

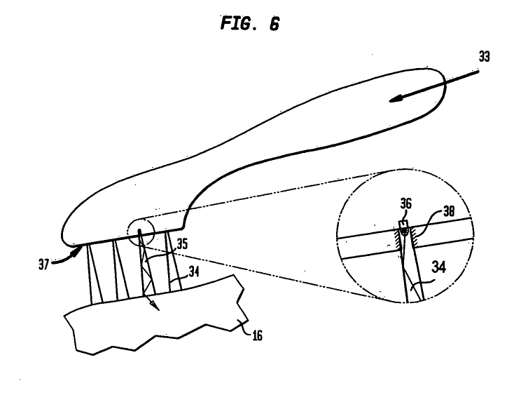

FIGS. 2 and 3 illustrate more preferred embodiments where bristles and projections respectively are used to deliver light from the radiation sources in wafer/package 14 to the patient's skin surface. To simplify these figures, heat sink 20 and handle 22 are not shown, however, a handle such as handle 22 (FIG. 1) or handgrip of some sort would normally be employed for each embodiment, and heat sink 20 could be employed if required. The nature and function of the bristles 26 shown in FIG. 2 have been previously discussed in some detail, as have the nature and function of the projections 30 shown in FIG. 3. Projections 30 can be molded into the housing of head 10″ and are preferably of an optically transparent material which may, for some embodiments, also have good heat transfer properties. To assure both good light and good heat transfer, there should be as little space as possible between wafer/package 14 and the projections. While projections 30 are shown as pointed in FIG. 3, and this is preferred for many applications, there are applications where a more rounded projection may be preferable. If some pressure is applied to head 10″, projections 30 will extend slightly below the skin surface to further enhance radiation delivery to a target area. Projections 30 can be designed and shaped so that, without contact with the skin, all or almost all light from light sources 14 is totally internally reflected and remains within the head, but, if the surface of a projection 30 has even slight optical contact with skin, light is coupled into the skin at that contact site. A lotion with the right refractive index can improve optical coupling. FIGS. 5A-5D show embodiments of this concept using the total internal reflection phenomena for projections and bristles. The light from light sources 31 with narrow divergence is normally completely reflected from distal end of projections 30 or transparent bristle 26 (FIGS. 5A and 5C) due to TIR because the refractive index of air is 1. However, if the distal end of projections 30 or transparent bristle 26 contacts skin 16 (FIGS. 5B and 5D), due to the high refractive index of skin n=1.4-1.5, most of the light is coupled into the skin. This concept can provide increased eye safety and comfort. In addition, back reflected light can be used as a signal for decreasing power to the light sources to save battery energy. The efficiency of light emitting applicator 10 can be increased by using a high reflecting front surface 32 to return light that is reflected from the skin back toward and into the skin.

TABLE 1
Preferred parameters of treatment with light
emitting applicator (LEA)

| Treatment condition or application | Wavelength, nm |
|---|---|
| Anti-aging | 400-2700 |
| Superficial vascular | 290-600 |
| | 1300-2700 |
| Deep vascular | 500-1300 |
| Pigmented lesion, de pigmentation | 290-1300 |
| Skin texture, stretch mark, scar, porous | 290-2700 |
| Deep wrinkle, elasticity | 500-1350 |
| Skin lifting | 600-1350 |
| Acne | 290-700, 900-1850 |
| Psoriasis | 290-600 |
| Hair growth control, | 400-1350 |
| PFB | 300-400, 450-1200 |
| Cellulite | 600-1350 |
| Skin cleaning | 290-700 |
| Odor | 290-1350 |
| Oiliness | 290-700, 900-1850 |
| Lotion delivery into the skin | 1200-20000 |
| Color lotion delivery into the skin | Spectrum of absorption |
| | of color center and |
| | 1200-20000 |
| Lotion with PDT effect on skin | Spectrum of absorption |
| condition including anti cancer effect | of photo sensitizer |
| ALA lotion with PDT effect on skin | 290-700 |
| condition including anti cancer effect | |
| Pain relief | 500-1350 |
| Muscular, joint treatment | 600-1350 |
| Blood, lymph, immune system | 290-1350 |
| Direct singlet oxygen generation | 1260-1280 |

Many additional embodiments of the invention are also possible; for example, a shower-head with LEA. FIG. 6 is a schematic of a shower-head LEA. Water 33 comes into the head through a handle and is distributed through holes 37 in water streams. Light sources 36 (for example, mini lamps or LEDs) are mounted close to each hole 37 so light can be coupled into the water stream exiting the hole, the water stream acting as a waveguide for better delivery of the light to the body. For this purpose, the internal surface of each hole can be coated with a high-reflection material.

LEA for delivering drug, lotion or other substance into the skin. The LEA can be built as a brush with bristles or projections transparent to light with wavelength(s) highly absorbed by the stratum cornea (water, lipid, keratinized cells). The distal end of each bristle/projection in contact with the skin can heat the stratum cornea to a high enough temperature to increase penetration of the lotion, drug or other substance through the stratum cornea. Since the area of high temperature in the cornea is relative small, and this area continues to move with the bristles/projections, this treatment can be painless. Treatment can be enhanced by combining an LEA with other actions, such as rotation or vibration of bristles, other mechanical vibration, magnetic field, electric field, acoustic field, etc.

A small electro-magnetic generator can be mounted into the LEA so that, during continuous movement of the LEA across of the skin, electrical energy can be generated drive and/or to pump the light sources.

The size and shape of each LEA can be optimized for the part of body on which it is to be used and the condition to be treated. Thus, a head LEA, comb LEA, facial LEA, beard LEA, breast LEA, leg LEA, body LEA, back LEA, underarm LEA, neck LEA etc. could be provided. The light sources could be retrofitted to an existing skin applicator, such as skin brushed, shower brushes, shave brushes, razors, tooth brushes, microabrasing applicator, massage device, lotion, gel, soaps, sponges, topical drug distributors, heat or cold applicator pad to form an LEA. For example, an array of light sources could be attached by Velcro, clip or other suitable means to a bath brush or other brush or body massager.

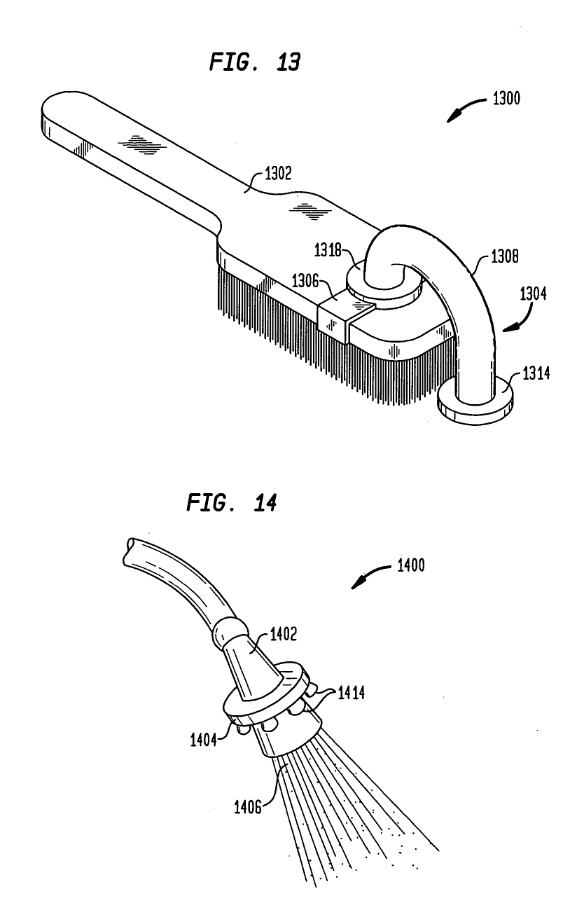

FIG. 13 illustrates another embodiment of the invention in which a retrofit or “snap-on” accessory phototreatment apparatus 1300 is joined to a skin surface treatment device, such as a brush 1302. Apparatus 1300 can include a housing 1304 with an attachment mechanism, e.g., one or more clips 1306 to secure the apparatus to the skin treatment device. Within the housing 1304 is at least one radiation source 1314 and, optionally, a power supply 1318 arranged, for example, as discussed above in connection with other figures. The housing can further include a flexible “gooseneck” linkage 1308 for adjustable disposition of the radiation source 1314.

FIG. 14 illustrates another retrofit apparatus 1400 for use in connection with a showerhead 1402 (or similar handheld bathing devices). Apparatus 1400 can include a securing band 1404 and at least one radiation source 1414 to deliver phototreatment concurrently with water delivery through nozzle 1406 of the showerhead.

A light emitting shaving brush may have both bristles for cream/gel distribution and/or skin massage and a light source with suitable power and wavelength. Light can be used for heating the cream and/or skin or hair shaft for better shaving, and can also function to control hair re-growth. The wavelength of the emitted light should be in the range of high absorption for melanin, water, lipid or shaft/stratum cornea cells. Systematic use of a light-emitting shaving brush can control skin sensitivity and skin sterilization. In this case, the wavelength should be selected from the range 290-1350 nm for cleaning of bacteria. This type of brush can be used for acne treatment and prevention. A light emitting shaving brush could also be used for control of hair growth. In this case, the wavelength should be selected from the range 400-1350 nm. Systematically using a light emitting shaving brush will be effective for slowing the hair growth rate and/or changing the hair texture and/or hair pigmentation. As a benefit, the interval between shaving can be increased due to hair growth delay. In addition, it may effectively treat/prevent razor bumps (PFB) and other skin problems caused by beard growth. Wavelengths in the range of about 300-400 nm can be used to softening the hair shaft and wavelengths in the range of about 600-1200 nm wavelengths can be used to suspend hair shaft growth, such as to prevent PFB. This brush may also be used for acne treatment and prevention. The light emitting shaving brush can also be used in combination with a light activated lotion, for example, a lotion with a photosensitizer or photosensitizer production compound such as ALA. The concentration of photosensitizer should be below a threshold of side effects from sun and other lightening systems, but above a threshold of photochemical effect on hair follicles, sebaceous glands or sebaceous follicles from a light emitting applicator. As a result, this treatment can be effective on hair growth, acne, skin oiliness, skin tone and skin texture.

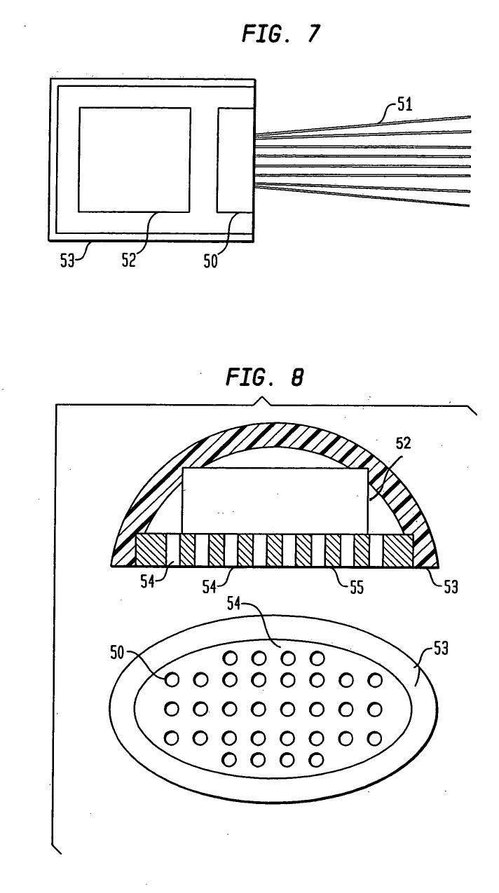

FIG. 7 is a schematic of one example of a light emitting shaving brush. Light from light sources 50 are partly or completely coupled into transparent bristles 51. Power supply 52 mounted to a handle 53 can be a rechargeable battery or a disposable battery. FIG. 8 is schematic of high efficiency applicator with both photo and thermal effect. Light sources 50 are mounted into a high thermo-conductive plate 54 (Cu, Al). The efficiency of light sources 50 can be 1-30% of the total electrical energy from power supply 52. The remaining 70-99% is heat energy from the light sources and power supply, this heat energy being coupled into plate 54 mounted to low thermo-conductive handle 53. Phase transfer material that can be used to cool light sources and electronics 52 can be placed between thermo conductive plate 54 and handle 53. Plate 54 should be designed with pins or other features, such as a heat pipe, that increase the contact surface with the phase transfer material. Temperature of the plate 54 during treatment should be close to the melting or vaporization temperature of the heat transfer material. During treatment, warmed plate 54 heats the superficial layer of the skin and/or any lotion on the skin. Light from the light sources penetrates into deeper skin layers for thermal treatment of deeper targets or for photochemical treatment. A vibrator can be positioned inside the applicator to massage the skin and increase light penetration into the skin. In another embodiment, the contact plate can be moveable or rotatable. This rotatable contact plate can be coupled to a micro-motor and used for skin micro abrasion and cleaning.

While the invention has been described above with reference to a number of embodiments, and variations on these embodiments have also been described, these embodiment and variations are by way of illustration only, and other embodiments and variations will be apparent to ones skilled in the art while still remaining within the spirit and scope of the invention. Therefore, the scope of the invention is to be limited only by the following claims.

1. Apparatus for treatment of a patient condition, comprising:
an applicator having a skin-contacting surface comprising at least one protuberance, and
at least one optical radiation source coupled to said applicator in a manner so as to, when activated, deliver optical radiation through said skin-contacting surface to a patient's skin in contact with said surface.

2. Apparatus as claimed in claim 1 wherein said applicator is in the form of a brush adapted to be moved over the patient's skin surface as radiation is applied thereto.

3. Apparatus as claimed in claim 1 wherein said applicator is in the form of a roller adapted to be moved over the patient's skin surface as radiation is applied thereto.

4. Apparatus as claimed in claim 1 wherein said skin-contacting surface has at least one protuberance selected from the group of projections and bristles extending therefrom.

5. Apparatus as claimed in claim 1 wherein said protuberance is adapted to apply a compressive force to the skin during use.

6. Apparatus as claimed in claim 1 wherein said radiation at the patient's skin surface is between approximately 1 mW/cm² and approximately 100 W/cm², the radiation depending at least on the condition being treated and the wavelength of the radiation.

7. Apparatus as claimed in claim 6 wherein said radiation at the patient's skin surface is between 10 mW/cm² and 10 W/cm².

8. Apparatus as claimed in claim 1 wherein said at least one optical radiation source is an array of optical radiation sources, each said source being mounted to deliver optical radiation through at least one corresponding protuberance.

9. Apparatus as claimed in claim 8 wherein each of the plurality of sources is mounted to deliver radiation through a corresponding protuberance.

10. Apparatus as claimed in claim 8 wherein a skin contacting end of each protuberance has total internal reflection for the radiation when not in contact with the patient's skin, but passes radiation to the patient's skin when in contact therewith.

11. Apparatus as claimed in claim 1 wherein said at least one optical radiation source is an array of semiconductor radiation-emitting elements.

12. Apparatus as claimed in claim 1 wherein the at least one optical radiation source is operable at different wavelengths to effect a desired treatment protocol.

13. Apparatus as claimed in claim 1 wherein the at least one optical radiation source is a continuous wave radiation source.

14. Apparatus as claimed in claim 1 further comprising a heat sink.

15. Apparatus as claimed in claim 14 including a handle for said apparatus which is adapted to be held by the operator when the apparatus is in use, said heat sink sinking heat from said at least one radiation source to said handle, heat from said handle being sinked to said operator's hand.

16. Apparatus as claimed in claim 11 including a detector of contact between said applicator and the patient's skin, and controls operative in response to said detector for permitting radiation to be applied from said at least one source to the patient's skin.

17. Apparatus as claimed in claim 1 wherein said apparatus includes a mechanism for applying a substance to the patient's skin as the skin is being irradiated.

18. Apparatus as claimed in claim 1 wherein said radiation sources are retrofitted to said applicator, and including a mechanism for attaching the sources to the applicator.

19. Apparatus as claimed in claim 1 wherein said at least one radiation source is part of said applicator.

20. Apparatus as claimed in claim 1 wherein said applicator is a hand-held unit.

21. Apparatus as claimed in claim 1 wherein said skin-contacting surface is formed of a plate having good thermal conducting properties, said at least one optical radiation source being mounted to said plate so that heat from said at least one source heats said plate, said heated plate thereby being adapted to heat a skin region during use.

22. Apparatus as claimed in claim 1 including a heat sink component in thermal contact with said at least one source, said component being adapted to be cooled prior to use of the apparatus.

23. Apparatus as claimed in claim 22 wherein said component undergoes a phase change when cooled, and returns to its initial phase when extracting heat from said at least one source.

24. Apparatus for treatment of a patient condition, comprising:
an applicator including at least one liquid delivery conduit for directing liquid onto a skin surface, and
at least one optical radiation source coupled to said applicator in a manner so as to, when activated, deliver optical radiation together with the liquid to the skin surface.

25. Apparatus as claimed in claim 24 wherein said applicator is a bath brush, water being applied through said applicator both for bathing or showering.

26. Apparatus as claimed in claim 25 wherein water is applied to also cool at least one radiation source.

27. Apparatus as claimed in claim 24 wherein said water is applied through openings in said surface to form water streams, and wherein radiation from said at least one source is also applied through said openings, said streams acting as wave guides for delivery of said radiation to the patient.

28. Apparatus as claimed in claim 24 wherein said applicator is shaped to fit a portion of the patient's body to be treated.

29. Apparatus as claimed in claim 24 including a mechanism for at least one of vibrating and otherwise stimulating the skin.

30. Apparatus as claimed in claim 24 wherein said radiation sources are retrofitted to said applicator, and including a mechanism for attaching the sources to the applicator.

31. Apparatus as claimed in claim 24 wherein said at least one radiation source is part of said applicator.

32. Apparatus as claimed in claim 24 wherein said applicator is a hand-held unit.

33. Apparatus as claimed in claim 24 wherein said skin-contacting surface is formed of a plate having good thermal conducting properties, said at least one optical radiation source being mounted to said plate so that heat extracted from said at least one source heats said plate, said heated plate thereby being adapted to heat a skin region during use.

34. Apparatus as claimed in claim 24 including a heat sink component in thermal contact with said at least one source, said component being adapted to be cooled prior to use of the apparatus.

35. Apparatus as claimed in claim 34 wherein said component undergoes a phase change when cooled, and returns to its initial phase when sinking heat from said at least one source.

36. Apparatus for treatment of a patient condition, comprising:
an applicator having a skin-contacting surface, and
at least one optical radiation source coupled to said applicator in a manner so as to, when activated, deliver optical radiation through said skin-contacting surface to a patient's skin in contact with said surface,
wherein the apparatus further comprises a mechanism for applying at least one of a magnetic field, an electric field and an acoustic field to the patient's skin.

37. Apparatus as claimed in claim 36 wherein said skin contacting surface is created such that it retro-reflects radiation reflected from the patient's skin back into the skin.

38. Apparatus as claimed in claim 36 including a generator activated by movement of the applicator over the patient's skin to generate electrical energy for the radiation sources.

39. Apparatus as claimed in claim 36 wherein said radiation sources are retrofitted to said applicator, and including a mechanism for attaching the sources to the applicator.

40. Apparatus as claimed in claim 36 wherein said at least one radiation source is part of said applicator.

41. Apparatus as claimed in claim 36 wherein said applicator is a hand-held unit.

42. Apparatus as claimed in claim 36 wherein said skin-contacting surface is formed of a plate having good thermal conducting properties, said at least one optical radiation source being mounted to said plate so that heat extracted from said at least one source heats said plate, said heated plate thereby being adapted to heat a skin region during use.

43. Apparatus as claimed in claim 36 including a heat sink component in thermal contact with said at least one source, said component being adapted to be cooled prior to use of the apparatus.

44. Apparatus as claimed in claim 43 wherein said component undergoes a phase change when cooled, and returns to its initial phase when sinking heat from said at least one source.

45. Apparatus for treatment of a patient condition, comprising:
a retrofit housing adapted to be joined to a skin-contacting device, and
at least one optical radiation source coupled to the retrofit housing in a manner so as to, when activated, deliver optical radiation to a skin surface concurrently with use of the skin-contacting device.

46. Apparatus as claimed in claim 45 wherein the skin-contacting device is in the form of a brush adapted to be moved over the patient's skin surface as radiation is applied thereto.

47. Apparatus as claimed in claim 45 wherein the skin-contacting device is in the form of a roller adapted to be moved over the patient's skin surface as radiation is applied thereto.

48. Apparatus as claimed in claim 45 wherein said skin-contacting surface has at least one protuberance selected from the group of projections and bristles extending therefrom.

49. Apparatus as claimed in claim 45 wherein said protuberance is adapted to apply a compressive force to the skin during use.

50. Apparatus as claimed in claim 45 wherein the skin-contacting device is in the form of a bath brush adapted to deliver water to a skin surface as radiation is applied thereto.

51. Apparatus as claimed in claim 45 wherein said radiation at the patient's skin surface is between approximately 1 mW/cm² and approximately 100 W/cm², the radiation depending at least on the condition being treated and the wavelength of the radiation.

52. Apparatus as claimed in claim 51 wherein said energy at the patient's skin surface is between 10 mW/cm² and 10 W/cm².

53. Apparatus as claimed in claim 45 wherein said at least one optical radiation source is an array of semiconductor radiation-emitting elements.

54. Apparatus as claimed in claim 45 wherein the at least one optical radiation source is operable at different wavelengths to effect a desired treatment protocol.

55. Apparatus for phototreatment substantially as shown and described.
